SEQUENCE LISTING (10 sequences)

SEQ ID NO: 1 - RNA, 84 nt, Homo sapiens
aggcugagguaguaguuuguacaguuugagggucuaugauaccacccgguacaggagauaacuguacaggccacugccuugcca

SEQ ID NO: 2 - RNA, 22 nt, Homo sapiens
ugagguaguaguuuguacaguu

SEQ ID NO: 3 - DNA, 22 bp, Artificial Sequence
  forward primer of VCAM-1 for quantitative PCR
tgccgagctaaattacacattg

SEQ ID NO: 4 - DNA, 21 bp, Artificial Sequence
  backward primer of VCAM-1 for quantitative PCR
ccttgtggagggatgtacaga

SEQ ID NO: 5 - DNA, 19 bp, Artificial Sequence
  forward primer of MCP-1 for quantitative PCR
cttctgggcctgctgttca

SEQ ID NO: 6 - DNA, 21 bp, Artificial Sequence
  backward primer of MCP-1 for quantitative PCR
ccagcctactcattgggatca

SEQ ID NO: 7 - DNA, 19 bp, Artificial Sequence
  forward primer of IL-6 for quantitative PCR
gacaactttggcattgtgg

SEQ ID NO: 8 - DNA, 19 bp, Artificial Sequence
  backward primer of IL-6 for quantitative PCR
atgcagggatgatgttctg

SEQ ID NO: 9 - DNA, 24 bp, Artificial Sequence
  forward primer of GAPDH for quantitative PCR
tctccctcacaatttccatcccag

SEQ ID NO: 10 - DNA, 24 bp, Artificial Sequence
  backward primer of GAPDH for quantitative PCR
gggtgcagcgaactttattgatgg

(19) United States
(12) Patent Application Publication
(10) Pub. No.: US 2015/0119443 A1
(43) Pub. Date: Apr. 30, 2015

(54) METHOD OF THERAPY AND DIAGNOSIS OF ENDOTHELIAL DYSFUNCTION

(71) Applicant: Kaohsiung Medical University, Kaohsiung (TW)

(72) Inventor: Suh-Hang Juo, Kaohsiung (TW)

(73) Assignee: Kaohsiung Medical University, Kaohsiung (TW)

(21) Appl. No.: 14/149,528

(22) Filed: Jan. 7, 2014

(30) Foreign Application Priority Data
Oct. 28, 2013 (TW) 102138939

(51) Int. Cl. C12N 15/113; C12Q 1/68

(52) U.S. Cl. CPC C12N 15/1136; C12Q 1/6883; C12N 2310/141; C12N 2320/30; C12Q 2600/178
USPC 514/44 A; 435/6.11

(57) ABSTRACT

The invention discloses a method of therapy of endothelial dysfunction, by administering microRNA let-7g to a subject in need, wherein the microRNA let-7g inhibits SMAD2 transcription factor from activation and translocation into nucleus, thereby decreasing monocyte cell adhesion, inflammation and thrombosis and increasing angiogenesis.

### BACKGROUND OF THE INVENTION

1. Field of the Invention

The present invention generally relates to a method of therapy and diagnosis of endothelial dysfunction and, more particularly, to a method of therapy and diagnosis of endothelial dysfunction in use of microRNA let-7g.

2. Description of the Related Art

Endothelium is a thin layer of cells lining the interior surface of blood vessels and lymphatic vessels which lines the entire circulatory system of organisms. In general, endothelial cells are involved in many aspects of vascular biology. For example, endothelial cells control blood pressure by regulating vasoconstriction and vasodilation. Endothelial cells acts as a barrier between the vessel lumen and surrounding tissues, controlling the passage of materials and the transit of white blood cells. Endothelial cells controls blood clotting by regulating thrombosis and fibrinolysis. Besides, endothelial cells also play an important role in inflammation and angiogenesis.

Endothelial dysfunction is a systemic pathological state of the endothelium and is due to endothelial senescence or perturbation of signal transduction of endothelial cells. Endothelial dysfunction is not only a key physiological event in the development of atherosclerosis and other related diseases, but also can result in vascular dementia, peripheral circulation dysfunction, sex dysfunction, retinopathy, diabetic vasculopathy, stroke and myocardial infraction, etc.

Generally, clinically used conventional therapeutic agents for vascular disorders include captopril, an angiotensin-converting enzyme (ACE) inhibitor for treating hypertension, and statin, HMG CoA reductase inhibitor to lower cholesterol level. However, the conventional therapeutic agents are not specifically for treating endothelial dysfunction and probably result in serious adverse effects such as hypotension, acute renal failure, sore muscle, rhabdomyolysis, liver dysfunction and hypolipidemia. Accordingly, it is still lack of a directly and effectively therapeutic strategy to improve endothelial function.

On the other hand, conventional diagnosis of endothelial dysfunction can be assessed using acetycholine via intravenous injection to induce endothelium-dependent dilation, followed by measuring the resultant relative increase of artery. The artery dilates in the absence of endothelial dysfunction while the artery obscurely dilates or even constricts in the presence of endothelial dysfunction. Nevertheless, the conventional diagnosis of endothelial dysfunction being an invasive way with high cost is therefore unsuited for application in clinical.

MicroRNAs, being a novel class of endogenous, small and non-coding RNAs, are widely found in organisms and generally control gene expression thereof. Precisely, microRNAs specifically target to particular genes and bind to their 3′ UTR region for up-regulating or down-regulating the gene expression of the particular genes. Inappropriate expression of microRNAs may affect the normal expression of their target genes, thereby playing an important role on many diseases including cancers, vascular disease, neurological diseases, degenerative diseases etc.

To date, microRNA let-7 has nine family members in human beings, and plays a crucial role in cell proliferation and cancer, wherein the pivotal role of microRNA let-7g in the regulation of Ras gene and liver cancer has been well-studied. MicroRNA-let7g is generally found in cells in the form of pre-let-7g as SEQ ID NO. 1, and which is further processed of enzymatic digestion to obtain a mature form of microRNA let-7g, as set forth in SEQ ID NO. 2. Recently, microRNA let-7g is used as a new therapy of cancer, and has been put to use in developing cancer related medication or treatment.

### SUMMARY OF THE INVENTION

It is therefore the objective of this invention to provide a method of therapy of endothelial dysfunction, by inhibiting TGF-β signaling pathway with microRNA let-7g, regulating monocyte cell adhesion, inflammation, thrombosis and angiogenesis, and further improving endothelial function.

It is another objective of this invention to provide a method of therapy of endothelial dysfunction, by delaying senescence of the endothelial cells with microRNA let-7g, and further preventing from senescence-induced endothelial dysfunction.

It is yet another objective of this invention to provide a method of diagnosis of endothelial dysfunction, by detecting microRNA let-7g level in serum samples to assess endothelial dysfunction.

A method of therapy of endothelial dysfunction comprises administering microRNA let-7g to a subject in need, wherein the microRNA let-7g inhibits SMAD2 transcription factor from activation and translocation into nucleus, thereby decreasing monocyte cell adhesion, inflammation and thrombosis and increasing angiogenesis. Moreover, the microRNA let-7g further promotes SIRT1 protein expression to prevent senescence-induced endothelial dysfunction.

A method of diagnosis of endothelial dysfunction comprises determining levels of microRNA let-7g in blood samples of organisms, in which the levels of microRNA let-7g is estimated in individuals with endothelial dysfunction.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention will become more fully understood from the detailed description given hereinafter and the accompanying drawings which are given by way of illustration only, and thus are not limitative of the present invention, and wherein:

FIG. 1 is a Western blotting showing microRNA let-7g inhibits SMAD2 protein expression.

FIG. 2 is an immunofluorescence staining showing microRNA let-7g inhibits SMAD2 from translocating into nucleus.

FIG. 3 is a bar chart showing microRNA let-7g inhibits VCAM-1 protein secretion.

FIG. 4A is a bar chart showing microRNA let-7g inhibits cytokine MCP-1 protein secretion.

FIG. 4B is a bar chart showing microRNA let-7g inhibits cytokine IL-6 protein secretion.

FIG. 5 is a bar chart showing microRNA let-7g inhibits PAI-1 protein expression.

FIG. 6 is an optical microphotograph showing microRNA let-7g promotes angiogenesis.

FIG. 7A is an immunohistochemistry staining showing microRNA let-7g inhibits SMAD2 protein from phosphorylation.

FIG. 7B is an immunohistochemistry staining showing microRNA let-7g inhibits PAI-1 protein expression.

FIG. 8A is a bar chart showing microRNA let-7g inhibits VCAM-1 mRNA expression.

FIG. 8B is a bar chart showing microRNA let-7g inhibits MCP-1 mRNA expression.

FIG. 8C is a bar chart showing microRNA let-7g inhibits IL-6 mRNA expression.

FIG. 9 a Western blot analysis showing microRNA let-7g promotes SIRT-1 protein expression.

In the various figures of the drawings, the same numerals designate the same or similar parts. Furthermore, when the term “first”, “second”, “third”, “fourth”, “inner”, “outer” “top”, “bottom” and similar terms are used hereinafter, it should be understood that these terms refer only to the structure shown in the drawings as it would appear to a person viewing the drawings, and are utilized only to facilitate describing the invention.

### DETAILED DESCRIPTION OF THE INVENTION

By inhibiting SMAD2 from activating and translocating into nucleus, microRNA let-7g can improve in endothelial function, such as a decrease of monocyte cell adhesion, anti-inflammation, anti-thrombosis and angiogenesis.

In order to prove that microRNA let-7g possesses effects on treating endothelial dysfunction, further experiments are performed as following:

Trial (A): MicroRNA Let-7g Inhibits HUVEC from Endothelial Dysfunction In Vitro

Human umbilical vein endothelial cells (HUVEC, purchased from Invitrogen, No. C-003-5C) is used in the trial (A). HUVEC are cultured in the medium containing M200 (Medium 200, purchased from Cascade Biologics, Portland, Oreg.), low serum growth supplement (LSGS, purchased from Invitrogen), 100 IU/mL of penicillin and 0.1 mg/mL of streptomycin. After subculturing for 4 to 6 generations, HUVEC are cultured in the incubator for 24 hours with temperature being 37° C., with CO₂ concentration being 5%, to drive HUVEC into quiescent stage.

Referring to Table 1, a let-7g analogue of let-7g, let-7g mimic (mirVana® miRNA mimicHsa-let-7g-5p, No. MC11758, purchased from Applied Biosystems), is used to demonstrate the expression and function of let-7g in HUVEC. Furthermore, mimic control (mirVana® miRNA Negative Control #1, No. 4464058, purchased from Applied Biosystems) is used as a negative control. In trial (A), Lipofectamine 2000 (purchased from Invitrogen) is co-transfected into HUVEC with 5 nM of the mimic control (group A1) or 5 nM of the let-7g mimic (group A2).

TABLE 1
Cell line and transfected miRNA used in the Trial (A)

| Groups | Cell line | Transfected miRNA |
|---|---|---|
| A1 | HUVEC | mimic control |
| A2 | HUVEC | let-7g mimic |

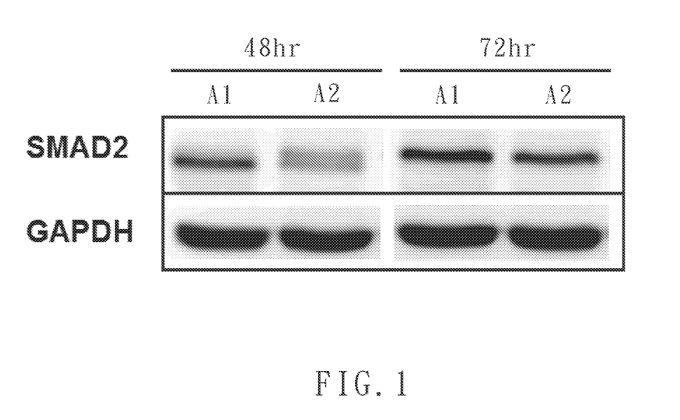

Cell lysis buffer (purchased from Cell signaling) containing protease inhibitor and phosphatase inhibitor cocktail (purchased from Calbiochem) is used to lyse microRNA transfected HUVEC. 40 μg of lysate is analyzed by 10% SDS-PAGE. Total proteins are transferred on PVDF membranes (purchased from Millipore), stained with an anti-total SMAD2 antibody (1:1000), an anti-pSMAD2 antibody (1:1000, specific to Serine 465 and 467 of SMAD2 protein) and an anti-GAPDH antibody (1:5000, as an internal control), and further stained with the horseradish peroxidase-conjugated secondary antibody recognizing the anti-total SMAD2 antibody, the anti-pSMAD2 antibody and the anti-GAPDH antibody (1:5000, purchased from Invitrogen). Finally, an enhanced chemiluminescent kit (ECL kit, purchased from Millipore) is used to detect the stained PVDF membranes. Intensities of each band are analyzed by ImageJ software. As shown in FIG. 1, the let-7g mimic (group A2) can inhibit not only SMAD2 protein expression but also phosphorylation on Serine 465 and Serine 467 (data not shown).

As a transcriptional factor, after phosphorylation by upstream signaling pathway, SMAD2 can translocate from cytoplasm into nucleus. Therefore, in order to demonstrate the activity of SMAD2, immunofluorescence staining is performed.

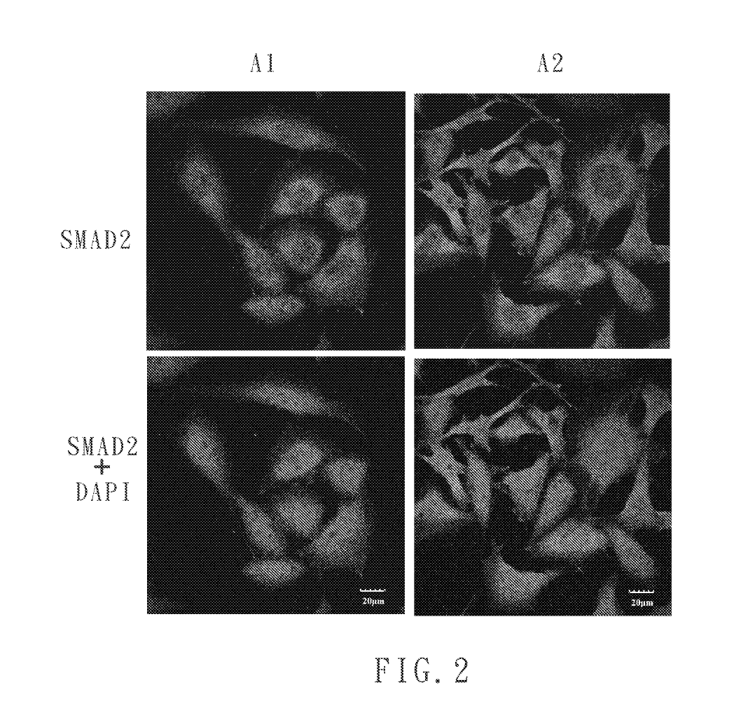

HUVEC is fixed by 4% paraformaldehyde and cell permeability is destroyed by 0.5% Triton X-100, thereby permitting the anti-total SMAD2 antibody (1:500, purchased from Cell signaling), AlexFluor 488 goat anti-rabbit capable of recognizing the anti-total SMAD2 antibody (1:500, purchased from Invitrogen) and DAPI (4′,6-diamidino-2-phenylinodole dihydrochloride, 1:1000, purchased from Invitrogen) enter into cytoplasm and nucleus. Stained total SMAD2 is observed by Fluoview™ FV1000 confocal microscopy (purchased from Olympus) with reference to FIG. 2. The let-7g mimic (group A2) is able to prevent SMAD2 from translocating into nucleus, thereby inhibit its downstream signaling pathway.

Being a member of TGF-β pathway, SMAD2 protein has several downstream targets including VCAM-1 (vascular cell adhesion molecule-1), MCP-1 (monocyte chemoattractant protein-1), IL-6 (interleukin-6) and PAI-1 (plasminogen activator inhibitor-1). All of VACM-1, MCP-1, IL-6 and PAI-1 involve in maintaining endothelial function, gene expression level of VCAM-1, MCP-1, IL-6 and PAI-1 are further measured as following.

HUVEC-culturing medium is collected at 0, 24, 48 or 72 hours, respectively. ELISA (enzyme-linked immunosorbent assay) kits are used to analyze protein expression of VCAM-1, MCP-1, IL-6 and PAI-1 according to the user guide of EnSpire™ Multimode Plate Reader (purchased from PerkinElmer). The ELISA kits for VCAM-1 and IL-6 are purchased from R & D and the ELISA kits for MCP-1 and PAI-1 are purchased from BD Biosciences.

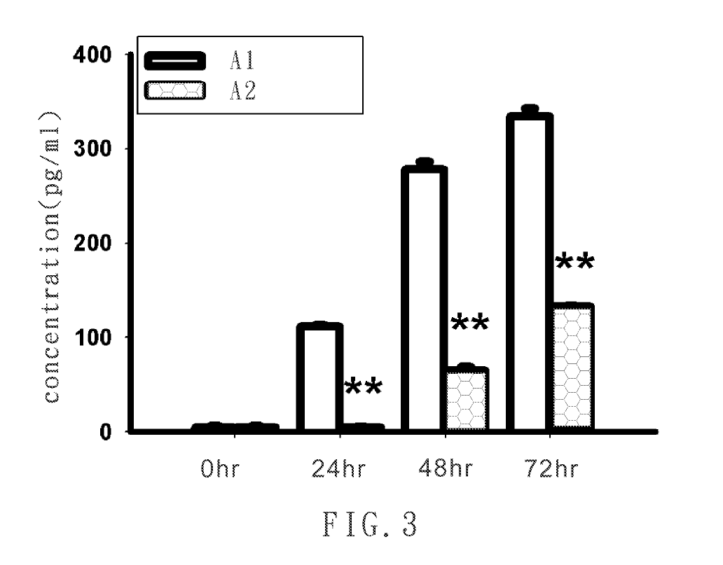

FIG. 3 indicates the let-7g mimic has ability to inhibit VCAM-1 protein expression in HUVEC. VCAM-1 is an important molecule in cell adhesion, therefore, through inhibition of VCAM-1 protein expression, the let-7g mimic prevents monocytes from adhering HUVEC by inhibiting VCAM-1 protein expression. That is, microRNA let-7 can prevent monocyte cell adhesion.

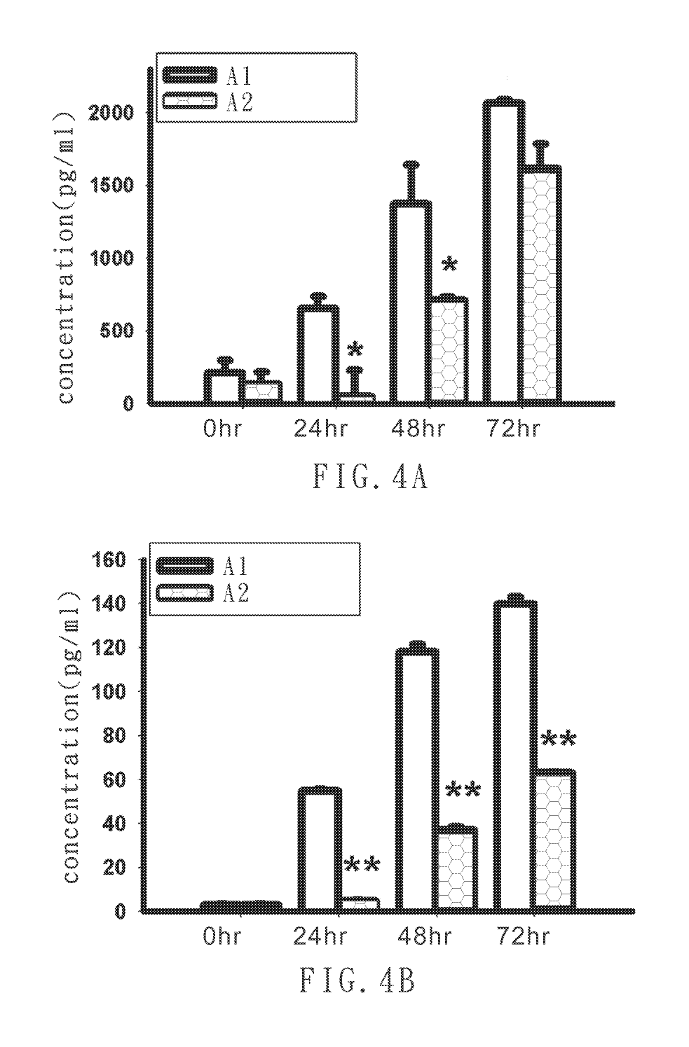

Moreover, FIGS. 4A and 4B show MCP-1 and IL-6 protein level in the HUVEC-culturing medium. The let-7g mimic (group A2) can inhibit HUVEC from secreting MCP-1 and IL-6 to the medium, decrease endothelial cell-permeable small, dense LDLs and prevent macrophages from gathering at endothelial cells, thereby avoiding the macrophages swallowing the small, dense LDLs and forming foam cells. That is, microRNA let-7g can reduce inflammation, foam cell formation and atherosclerosis.

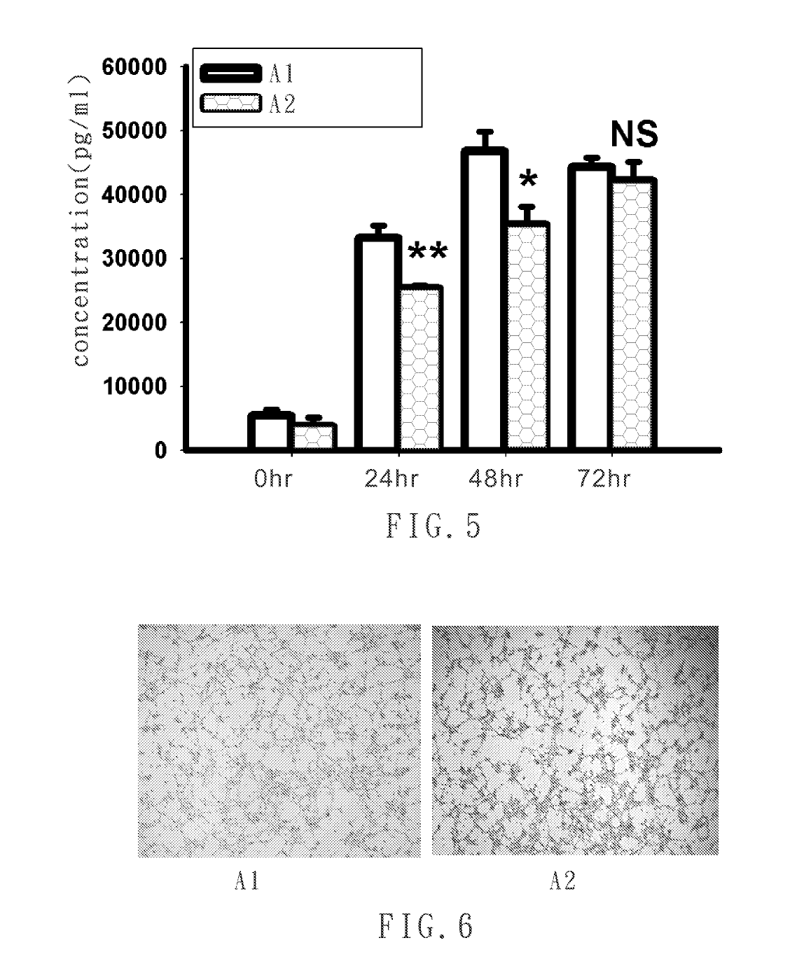

FIG. 5 shows secreted PAI-1 protein level in the HUVEC-culturing medium. The let-7g mimic (group A2) can prevent HUVEC from secreting PAI-1 protein into the medium. The let-7g mimic promotes plasminogen transform to plasmin, thereby decreasing fibrin clot and preventing from blood clotting. That is, microRNA let-7g can prevent from blood clotting.

Besides, pre-warmed matrigels (at 37° C., purchased from BD) are coated on a 24-well plate. The mimic control- or the let-7g mimic-transfected HUVEC for 24 hours is inoculated in the 24-well plate with a density being 70000 cells per well, respectively. After culturing for 5 hours, phase-contrast images (25× and 50×) are shoot by an optical microscopy. Referring to FIG. 6, the let-7g mimic (group A2) can promote HUVEC tubule formation. That is, microRNA let-7g can improve in angiogenesis.

Trial (B): MicroRNA Let-7g Inhibits ApoE-KO Mice from Endothelial Dysfunction In Vivo

With reference to Table 2, ApoE-KO male mice (apolipoprotein E-knock out mice, purchased from Jackson Laboratory) are used in the trial (B). The mice are kept on normal diet until 8 weeks, followed by feeding with high fat diet containing 0.15% cholesterol (purchased from Testdiet® 57BD) to induce fatty streak lesion in cervical artery. Lentivirus empty vector (group B1, pCDH-CMV-MCS-EF1-GreenPuro, purchased from SBI) or microRNA let-7g-expressing lentivirus vector (group B2) are injected into the tail vein of the ApoE-KO mice, respectively, wherein the let-7g-expressing lentivirus vector is dissolved in 0.2 mL of phosphate buffer with a dosage being 1×10⁷ TU. As such performance, the let-7g level of group B2 is 2.6-fold compared with group B1 (data not shown).

TABLE 2
Animal model and transfected miRNA used in the trail (B)

| Groups | Animal model | Transfected miRNA |
|---|---|---|
| B1 | ApoE-KO mice | Empty vector |
| B2 | ApoE-KO mice | let-7g |

The ApoE-KO mice are sacrificed 12 weeks later after transfection. Carotid arteries of groups B1 and B2 are fixed in Sakura Tissue-Tek® O.C.T compound. The fixed carotid arteries are sliced in a thickness being 8 The slices are stained with the anti-pSMAD2 antibody (1, 2000, purchased from Millipore) or an anti-PAI-1 antibody (1:100, purchased from Novus Biologicals), and further stained with IHC Select™ kit (purchased from Millipore). Finally, the staining results are analyzed by TissueFAXS system (purchased from TissueGnostics GmbH, Australia).

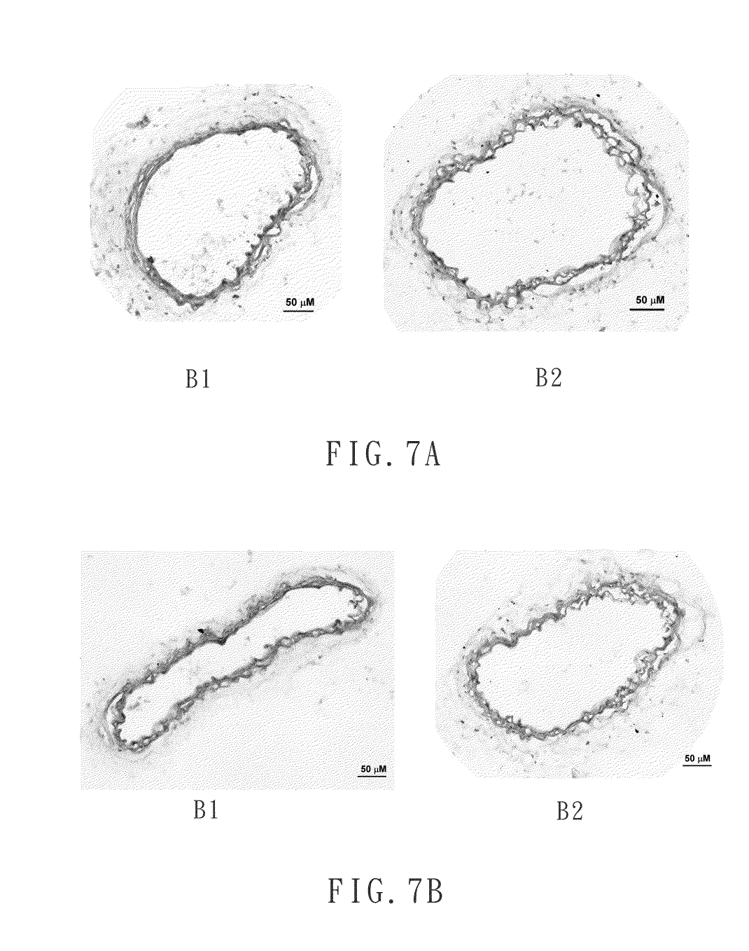

Referring to FIGS. 7A and 7B, pSMAD2 protein expression level decreases in group B2, the same as the results shown in trial (A). That is, overexpression of microRNA let-7g can inhibit phosphorylation of SMAD2 protein, thereby inhibiting downstream signaling pathway related to SMAD2 protein. Moreover, overexpression of microRNA let-7g can also inhibit PAI-1 protein expression, thereby preventing from thrombosis.

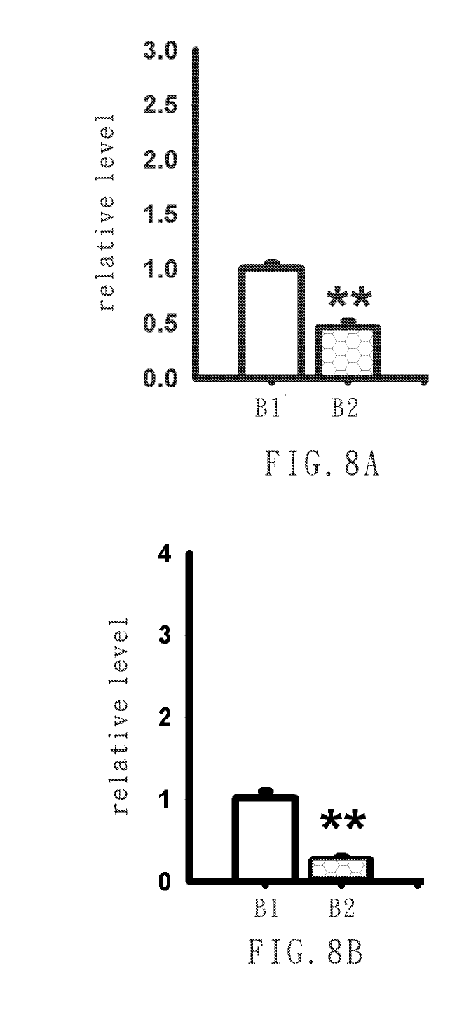

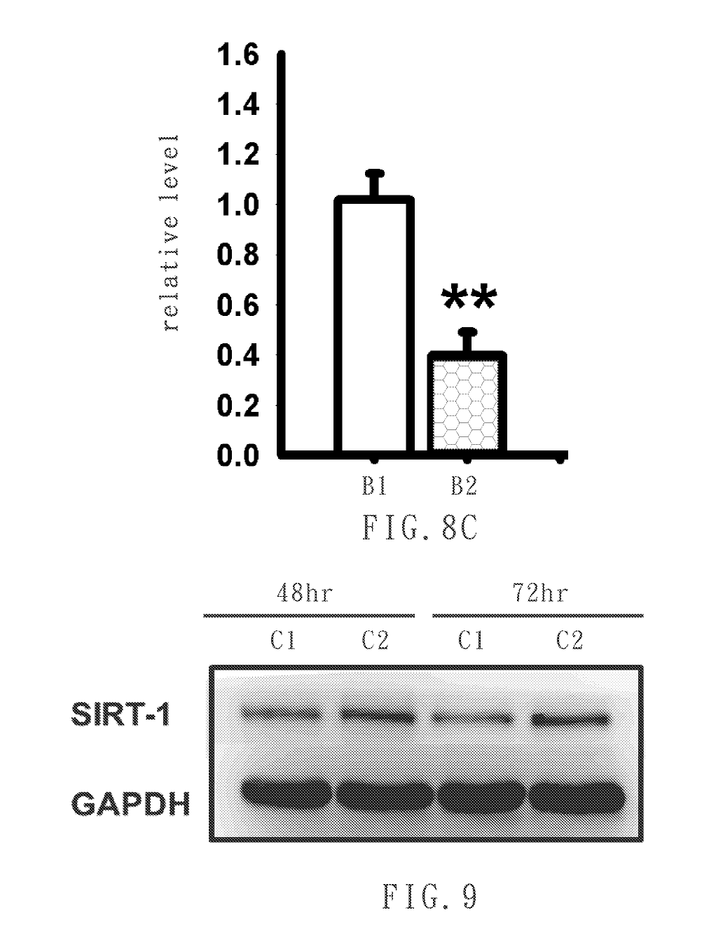

With respect to FIGS. 8A, 8B and 8C, serum samples of groups B1 and B2 are analyzed by quantitative PCR. That is, total RNA of the serum samples are reverse transcripted to cDNA, and primer pairs as set forth in SEQ ID NOs: 3 to 8 are used to quantitative analyze VCAM-1, MCP-1 and IL-6 mRNA expression. Furthermore, as an internal control, a primer pair as set forth in SEQ ID NOs: 9 and 10 are used to quantitative analyze GAPDH mRNA expression. As a result, by inhibiting SMAD2 phosphorylation, microRNA let-7g can inhibit mRNA expression of TGF-β signaling pathway members including VCAM-1, MCP-1 and IL-6, thereby decreasing monocyte cell adhesion and inflammation, the same as the results shown in trial (A).

Accordingly, by inhibiting protein expression and phosphorylation of SMAD2 protein, microRNA let-7g is able to affect TGF-β signaling pathway and regulate mRNA and protein expression of its downstream molecules including VCAM-1, MCP-1, IL-6 and PAI-1, thereby preventing monocytes from adhering to endothelial cells, and preventing the transformation of macrophages to foam cells. MicroRNA let-7g has ability to improve in endothelial function and further lowers possibilities of atherosclerosis and other related diseases.

Trial (C): MicroRNA Let-7g Delays Senescence of HUVEC In Vitro

With reference to Table 3, HUVEC transfected with 5 nM of the mimic control (group C1) or 5 nM of the let-7g mimic (group C2) are used in the trial (C).

TABLE 3
Cell line and transfected miRNA used in the trail (C)

| Groups | Cell line | Transfected miRNA |
|---|---|---|
| C1 | HUVEC | mimic control |
| C2 | HUVEC | let-7g mimic |

HUVEC of groups C1 and C2 are lysed and analyzed with Western blotting, wherein an anti-SIRT-1 antibody (1:1000, purchased from Cell signaling), the anti-GAPDH antibody (1:5000, purchased from Cell signaling, used as a negative control) and horseradish peroxidase-conjugated secondary antibody recognizing the anti-SIRT-1 antibody and the anti-GAPDH antibody (1:5000, purchased from Invitrogen) are used. As shown in FIG. 9, the let-7g mimic can elevate SIRT-1 protein expression. That is, microRNA let-7g can delay senescence of endothelial cells.

Trial (D): MicroRNA Let-7g Acts as a Diagnostic Marker for Endothelial Dysfunction in Humans

In trail (D), serum samples of 35 human subjects are analyzed by quantitative PCR. Total RNA extracted from the serum samples are reverse transcripted to cDNA, and further amplified by a commercial TaqManmiRNAexpression assay (hsa-let-7g, ID: 002282, with an internal control being RNU6B, ID: 001093). The let-7g ratio of each stroke subject to the mean of all stroke subjects is calculated by 2^{-ΔΔCt} where ΔΔCt is calculated by ΔCt of individual subject minus the mean of ΔCt from all stroke patients. ΔCt was referred to Ct_{let-7g}-Ct_{U6B}. The results are shown in Table 4.

TABLE 4
Serum let-7g ratio, plasma PAI-1 and ADMA levels in the trail (D)

| | Serum let-7g ratio | Plasma PAI-1 level | Plasma ADMA |
|---|---|---|---|
| Groups | (2^{-ΔΔCt}) | (ng/mL) | level (μmol/mL) |
| D1 | 0.48 ± 0.27 | 5.61 ± 3.24 | 0.60 ± 0.19 |
| D2 | 2.73 ± 1.36 | 3.70 ± 1.85 | 0.57 ± 0.21 |

Referring to Table 4, the 35 human subjects are separated into group D1 with lower serum let-7g ratio and group D2 with higher serum let-7g ratio. Plasma PAI-1 and ADMA levels of groups Dl and D2 are analyzed using ELISA kit according to the user guide of EnSpire™ Multimode Plate Reader (purchased from PerkinElmer). The ELISA kits for PAI-1 purchased from R&D, Minneapolis, Minn. and the ELISA kit for ADMA (asymmetric dimethylarginine) is purchased from DLD (dignostika GmbH, Germany). As a result, group D1 with lower serum let-7g ratio has a higher plasma PAI-1 protein level, meaning that the human subjects of group D1 are with endothelial dysfunction. Otherwise, group D2 with higher serum let-7g ratio has a lower plasma PAI-1 protein level, meaning that the human subjects of group D2 have normal endothelial function.

Accordingly, microRNA let-7g can indeed be used as a diagnostic marker in clinical diagnosis of endothelial dysfunction. By determining let-7g levels in serum samples, people at high risk to endothelial dysfunction can be easily and specifically noticed, so as to facilitate the identification and clinical treatment of endothelial dysfunction subjects, preventing from developing into atherosclerosis, vascular dementia, peripheral circulation dysfunction, sex dysfunction, retinopathy, stroke or myocardial infraction. That is, microRNA let-7g is capable of applying to development of novel diagnosis for endothelial dysfunction, being an assessment of those related diseases, thereby advancing efficiency and quality of diagnosis for the related diseases.

In summary, the method of therapy and diagnosis of endothelial dysfunction in the present invention provides a new strategy in clinical therapy and diagnosis of endothelial dysfunction, and not only can meet the need of rapid, sensitive and specific diagnostic assay of endothelial dysfunction, but also can effectively block the pathogenesis of endothelial dysfunction by administrating microRNA let-7g. Hence, according to the present invention, the quality of clinical therapy and diagnosis of endothelial dysfunction will be significantly improved.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A method of therapy of endothelial dysfunction, by administering microRNA let-7g to a subject in need, wherein the microRNA let-7g inhibits SMAD2 transcription factor from activation and translocation into nucleus, thereby decreasing monocyte cell adhesion, inflammation and thrombosis and increasing angiogenesis.

2. A method of therapy of endothelial dysfunction as claimed in claim 1, wherein the microRNA let-7g promotes SIRT1 protein expression to prevent senescence-induced endothelial dysfunction.

3. A method of diagnosis of endothelial dysfunction, comprising determining levels of microRNA let-7g in blood samples of organisms, in which the levels of microRNA let-7g are estimated in individuals to evaluate endothelial function.

4. A use of microRNA let-7g, by preventing transcription factor SMAD2 from activating and translocating into nucleus, to decrease monocyte cell adhesion, inflammation and thrombosis and to increase angiogenesis, thereby improving in endothelial function.

5. A use of microRNA let-7g, by promoting SIRT1 protein expression to inhibit senescence-induced endothelial dysfunction.

6. A use of microRNA let-7g as a diagnostic marker of endothelial dysfunction, wherein levels of microRNA let-7g in blood samples of organisms is determined to be the diagnostic marker of endothelial dysfunction.
